(19) United States
(12) Patent Application Publication
(10) Pub. No.: US 2008/0044855 A1
(43) Pub. Date: Feb. 21, 2008

(54) System for regulated and enhanced baculovirus mediated transient transgene expression in mammalian cells

(76) Inventors: Yan Xu, Montreal (CA); Cynthia Elias, Montreal (CA); Alaka Mullick, Montreal West (CA); Bernard Massie, Laval (CA); Barbara Jardin, Montreal (CA)

Correspondence Address: NATIONAL RESEARCH COUNCIL OF CANADA;1200 MONTREAL ROAD, BLDG M-58, ROOM EG12, OTTAWA, ONTARIO, K1A 0R6, CA

(21) Appl. No.: 11/723,610

(22) Filed: Mar. 21, 2007

Related U.S. Application Data
(60) Provisional application No. 60/784,483.
(60) Provisional application No. 60/798,748, filed on May 9, 2006.

(51) Int. Cl. C12P 21/06; C12N 5/06; C12N 15/86

(52) U.S. Cl. 435/69.1; 435/456; 435/358

(57) ABSTRACT

A baculovirus-based expression system under control of CR5 promoter improves expression of transgenic nucleic acid molecules in mammalian cells. It also provides a platform for regulated expression of transgenic nucleic acid molecules in mammalian cells.

### CROSS-REFERENCE TO RELATED APPLICATIONS

This application claims the benefit of U.S. Provisional applications U.S. Ser. No. 60/784,483 filed Mar. 22, 2006 and U.S. Ser. No. 60/798,748 filed May 9, 2006, the entire contents of both of which are herein incorporated by reference.

### FIELD OF THE INVENTION

The present invention relates to expression of transgenes in mammalian cells, particularly to vectors for introducing transgenes into mammalian cells.

### BACKGROUND OF THE INVENTION

The baculovirus expression system described first in the 1980s (Smith et al, 1983) has been successfully used since then for the production of several recombinant proteins both in industry and academia. This system has some inherent advantages, not only from a safety perspective but also due to the large capacity of the genome, the ease of production in insect cells and scalability (Kost and Condereay, 2002; Kost et al, 2005). The development of baculoviruses containing promoters active in mammalian cells opened up the possibilities of using these versatile vectors for gene delivery in mammalian cells (Hoffman et al, 1995; Boyce and Bucher, 1996; Condreay et al, 1999). Mammalian cells are increasingly being used for production of recombinant proteins to synthesize new therapeutics and also for numerous studies on gene and protein structure and function. The BacMam system is an example of an elegant baculovirus-based method for gene delivery into the mammalian cells which can thus have a significant impact, in the domains of protein production and in functional genomics.

For protein production, the Chinese Hamster Ovary (CHO) cell line is the cell line of choice. However, it is not very amenable to lipid/non-viral transient transfection methods creating a major bottleneck for rapid large-scale protein production. The expression of proteins using the stable transfection method is time consuming and entails the screening of hundreds of clones to obtain a cell line expressing the protein at high levels. Therefore, there is an overwhelming drive to develop a novel system to rapidly and efficiently express transgenes at high levels in CHO cells.

The most commonly used promoter for baculovirus mediated gene expression in mammalian cells is the cytomegalovirus (CMV) based promoter. However, the CMV promoter is not ideal for CHO cells due to its low activity and a number of strategies have been employed to augment promoter activity in these cells to achieve high-level protein production in a transient system. These include treatment with butyrate, a non-specific inhibitor of histone deacetylase (Conderay et al, 1999), incubation of cells at a lower temperature (Ramos et al, 2002) and using adenovirus E1a (Ramos et al, 2002) to activate the CMV promoter.

For functional genomics, it is important to carry out the studies in a cell type that is relevant for the function of the protein being studied. A major limitation to this is the low transfection efficiencies of several cell lines. The BacMam system is an attractive alternative, especially if the phenotype being assayed is apparent in a time frame of days rather than months. However, the use of a promoter with strong activity in the cell line of choice is a prerequisite for keeping viral load to a minimum. In addition, for functional analysis an inducible system has the advantage of permitting the comparison of the same cells before and after the addition of an inducer. To our knowledge, no inducible systems in the baculovirus system have been reported.

An expression cassette for mammalian cells (Massie et al, 2002) has been developed that has demonstrated high-level activity and tight regulation in stable cell lines (Mullick et al, 2006). In this cassette, expression of the gene of interest is regulated by a cumate-inducible promoter, CR5. CR5 can be activated by a chimeric transactivator rcTA in the presence of a small molecule, cumate.

There remains a need in the art for a baculovirus-based expression system, particularly one that is inducible, that can efficiently enhance expression of transgenes in mammalian cells.

### SUMMARY OF THE INVENTION

It has now been found that a baculovirus-based expression system under control of CR5 promoter unexpectedly and significantly improves expression of transgenic nucleic acid molecules in mammalian cells.

In an aspect of the invention there is provided an expression vector comprising a recombinant baculovirus operatively linked to a CR5 promoter.

In another aspect of the invention, there is provided a construct comprising the vector of the present invention operatively linked to a nucleic acid molecule of interest.

In yet another aspect of the invention, there is provided a method of expressing a nucleic acid molecule of interest in a mammalian cell comprising transducing the mammalian cell by a construct of the present invention in the presence of an activator for the CR5 promoter.

In still yet another aspect of the invention, there is provided a host mammalian cell transduced by the construct of the present invention.

Recombinant baculovirus expression systems are generally known in the art and these can be adapted to the present invention by first cutting out the undesired promoter to obtain an empty vector and then sub-cloning the CR5 promoter into the empty vector to obtain a vector of the present invention. To obtain a construct of the present invention, the nucleic acid molecule of interest is then sub-cloned into the vector, preferably downstream of the CR5 promoter. Examples of recombinant baculovirus expression systems are described in U.S. Pat. No. 5,731,182 issued Mar. 24, 1998 to Boyce, the disclosure of which is herein incorporated by reference in its entirety.

The CR5 promoter is described in commonly owned US patent publication 2004/0205834 published Oct. 14, 2004, the disclosure of which is herein incorporated by reference in it entirety. In brief, the CR5 promoter is a recombinant DNA molecule comprising a mammalian promoter sequence having a TATA element and at least one coding sequence of a cumate operator (CuO) operably linked to the mammalian promoter sequence positioned 3′ to the TATA element. The mammalian promoter sequence that forms part of the CR5 promoter may be, for example, CMV, VIP, tk, HSP, MLP or MMTV promoters. Preferably, the CR5 promoter comprises six CuO coding sequences operably linked to the mammalian promoter sequence.

The CR5 promoter may be activated by the presence of an activator, for example a chimeric transactivator (e.g. rcTA). The transactivator may be stably expressed in the mammalian cell and expression induced by the presence of an inducer; the expression of the transactivator in the mammalian cell may be constitutively high, obviating the need for the inducer; and/or, the transactivator may be transduced into the mammalian cell by a vector. Where an inducer is used, the inducer may comprise, for example, cumate, butyrate, dimethyl-p-aminobenzoic acid (DM PABA), trimethyl cumate, ethylbenzoate, a salt thereof or a combination thereof. Cumate, butyrate or a combination thereof is preferred.

The nucleic acid molecule of interest may be, for example, an antisense inhibitor of gene expression, a nucleic acid coding for a protein, or any other nucleic acid molecule for which expression is desired in the mammalian cell. Preferably, the nucleic acid molecule encodes a protein. In a construct of the present invention, the nucleic acid molecule of interest is operably linked to and lies 3′ to the CR5 promoter.

Advantageously, the present invention permits using baculovirus at lower multiplicity of infection (MOI) while maintaining high levels of transgene expression in mammalian cells in comparison to prior art baculovirus expression systems. For example, the CR5 promoter used in the present invention is unexpectedly stronger than prior art CMV5 promoter+butyrate system, and the CMV5 promoter is 10-fold stronger than the ordinary CMV promoter (Massie et al, 1998). Thus, the CR5 promoter is stronger than heretofore known promoters in BacMam expression systems.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIGS. 1A and 1B depict a comparison of baculovirus transduction under control of CR5 promoter versus CMV5 promoter for the expression of green fluorescent protein (GFP) in CHO cells;

FIG. 2 depicts expression of a toxic protein (Rep) in CHO cells by a baculovirus expression system under control of CR5 promoter;

FIG. 3A depicts EGFR expression on the surfaces of CHO-CymR-rcTA/10-35 and CHO-rcTA/227 cells;

FIG. 3B depicts FACS analysis of CHO-CymR-rcTA-EGFR and CHO-rcTA-EGFR;

FIG. 3C depicts western blots of EGFR and activated EGFR;

FIG. 4 depicts GFP expression in CHO cells co-transduced with BacMam-CR5-GFP and BacMam-CMV5-rcTA; and,

FIG. 5 depicts expression of the antibody B43 in CHO-CymR-rcTA/10-35 cell lines transduced with BacMam-CR5-b43 under cumate induction.

### DESCRIPTION OF PREFERRED EMBODIMENTS

Materials and Methods:

Cell Culture: CHO cells and the stable CHO cell lines (CHO-CymR-rcTA/10-35 and CHO-rcTA/227) were cultured in CD-CHO medium (Gibco, Grand Island, N.Y.) supplemented with 4 mM L-glutamine (Gibco), 1×HT supplement (0.1 mM sodium hypoxanthine and 0.016 mM thymidine) (Gibco), and 1× dextran. The cells were maintained in a humidified incubator at 37° C. with 5% CO₂. Sf9 cells (Spodoptera frugiperda) were maintained at 27° C. in shaker flasks, agitated at 110-120 rpm. Sf9 cells were grown in Sf900-II medium (Gibco, Invitrogen Life Technologies, Carlsbad, Calif.). All fine chemicals were from Sigma Aldrich, St. Louis, Mo.

Plasmid Construction: To construct transfer vectors for generating recombinant baculovirus with a mammalian promoter, the baculovirus polyhedrin (polh) promoter in pVL-1393 was replaced with CR5 promoter (pVL-3-CR5-GFP, pVL-3-CR5-EGFR and pVL-3-CR5-Rep) or CMV5 promoter (pVL-3-CMV5-GFP, pVL-3-CMV5-rCTA).

pVL-3-CR5-GFP, pVL-3-CR5-EGFR and pVL-3-CR5-Rep were constructed in two steps. First, plasmid pAdCR5-GFP (Mullick et al, 2006) was digested by restriction enzyme AflII, filled in by Klenow fragments, and digested again by BamHI to obtain the DNA fragment containing the CR5 promoter. Meanwhile, the baculovirus plasmid pVL-1393 bearing the baculovirus polyhedrin (polh) promoter was digested by EcoRV and BamHI to remove the polh promoter. The CR5 promoter was then subcloned into the digested vector pVL-1393 to form the desired BacMam plasmid pVL-3-CR5. The second step was to insert the coding sequences for GFP, EGFR and Rep downstream of CR5 in the plasmid pVL-3-CR5. The GFP cDNA fragment, flanked by BamHI and NotI restriction sites, was obtained by PCR amplification from plasmid pAdCR5-GFP. The EGFR cDNA was a kind gift from Dr. Maria Jaramillo. The Rep cDNA fragment was released from pAAV-RC (Stratagene), and then subcloned into a BglII-digested pVL-3-CR5 plasmid.

Construction of BacMam plasmids pVL-3-CMV5-GFP and pVL-3-CMV5-rCTA was also carried out in two stages, first to get an empty vector and second to subclone the desired genes into it. In order to obtain a baculovirus vector without any promoter, the plasmid pVL-1393 was cut by BamHI, filled in by Klenow fragments and cut again by EcoRV to remove the virus polyhedrin promoter. The resultant vector with two blunt ends was then self-ligated and re-digested by either BglII (pVL-3-CMV5-GFP) or SmaI for the next subcloning. A cDNA fragment containing CMV5-GFP sequences was released from pAdCMV5-GFP (Massie et al, 1998) by BglII digestion and cloned into BglII-digested vector to generate pVL-3-CMV5-GFP. Similarly, a cDNA fragment containing CMV5-rcTA sequences was released from pAd3-4C (Xu et al, 2004), by digestion with AflII and PmeI and cloned into a SmaI-digested vector to generate pVL-3-CMV5-rCTA.

Transfer vector pVL-b43 under the control of the CR5 promoter coding for an antibody IgG was created by cloning the entire sequence of b43 (the heavy and light chains) into the baculovirus.

Baculovirus Construction: Recombinant baculoviruses were generated in Sf-9 insect cells using homologous recombination with the linearized baculovirus genome Baculogold (Pharmingen). Approximately 10⁶ cells were plated onto each well of a 6-well plate. The transfection mixture comprised 1 μg of the transfer vector DNA (pVLCMV5-GFP, pVL-CR5-GFP, pCMV5-rcTA, pVL-CR5-EGFR, pVL-CR5-Rep or pVL-CR5-b43), 0.2 μg of the baculovirus DNA and the transfection reagent Cellfectin™ (Invitrogen Life Technologies, Carlsbad, Calif.) in IPL-41 basal medium. This was incubated for 45 minutes prior to addition to the cells. The transfection mixture was allowed to incubate with the cells for 5 h at 27° C., followed by replacement of the mixture with fresh medium (SF900 II). These cultures were further incubated at 27° C., supernatants were collected at 96 h post transfection, centrifuged (4° C., 1000 g, 10 min), filtered through a 0.22 μm and stored at 4° C. These were designated as passage one virus stock. Larger volumes of virus stock were made by amplification in shake flask cultures of Sf9 cells. Briefly this was done as follows. Sf9 cells were grown to mid-exponential phase and diluted to 2−2.5×10⁶ cells/ml with fresh medium. The cultures were infected with passage one virus stock at a MOI of 0.1-1. The total and viable cell densities and cell size were measured (using the automated Trypan Blue exclusion method, Cedex, Innovatis, Bielfeld, Germany) during the infection process and the supernatant was harvested by centrifugation when viability was less the 80-90%.

Baculovirus Quantification: Baculovirus stocks were quantified using a DNA staining method previously described by Shen et. al, 2002. Briefly, the virus particles were permealized and the viral DNA was labeled with the SYBR green fluorescent dye (Molecular Probes, Eugene, Oreg.). The viral particles were estimated by counting the labeled DNA using a FACS (EPICS XL-MCL, Beckman Coulter, Miami, Fla.) at 550 long pass dichroic and a 525 nm band pass filter. Calibration was performed using standard fluorospheres.

Transduction of CHO Cells with Baculovirus: CHO, CHOCymR-rcTA/10-35 and CHOrcTA/227 cells were grown to 2×10⁶ cells/ml. At this point, the growth was arrested by decreasing the temperature from 37° C. to 30° C. The cells were maintained at this temperature for 10-16 hr. Transduction was initiated by the addition of the baculovirus at the desired MOI. Sixteen hours later the cells were induced with 40 μg/ml of cumate.

Analytical Methods: Total and viable cell densities and cell size were measured using the automated Trypan Blue exclusion method (Cedex, Innovatis, Bielfield, Germany). Concentrations of glucose, glutamine, lactate and ammonia were measured using the Bioanalytical system (YSI 7100 MBS, Yellow Springs, Ohio). The proteins produced (Rep and EGFR) were analyzed by SDS-PAGE and Western blot methods.

Western Blot Analysis: Cell pellets were extracted in Lysis buffer (50 mM Hepes pH 7.4, 150 mM NaCl, 1% Thesit and 0.5% deoxycholate). Following the addition of the lysis buffer, the samples were incubated on ice and periodically vortexed over a 30 minute period. The soluble fraction was collected by centrifugation (4° C., 10-minute, 10000×g) and was mixed with sample buffer containing DTT and was heated at 95° C. for 5 minutes. Samples were run in MOPS buffer on a 4-12% NU-PAGE gradient gel (Invitrogen). Protein was transferred onto nitrocellulose for 1 hr period. The blots were probed with rabbit anti-human EGFR (SantaCruz Biotechnologies, Santa Cruz, Calif.) or anti-phosphotyrosine 4G-10 (Upstate, Charlottesville, Va. USA) to detect EGFR expression and its autophosphorylation activity. Antibody binding was detected using the chemiluminescence kit (Boehringer Mannheim, Mannheim, Germany) and visualized with the Kodak imager System.

FACS-based Assay for Cell Surface EGFR Expression: 1.5×10⁶ cells per well (in 6-well plates) of CHOCymR-rCTA/10-35 and CHO-rcTA/227 cells were infected by baculovirus pVL-CR5-EGFR at an MOI of 100 and incubated at 30° C. in 5% CO₂ for 5 hours. Expression of EGFR was induced by the addition of 30 μg/ml cumate. After 48 h the cells were harvested and the cell pellet was re-suspended in 100 μl of 1×PBS containing 1 μg of primary antibody against EGFR (EGFR mAB, clone 225, purified from hybridoma (ATCC)) and incubated for 60 minutes at room temperature. The cells were then washed three times and re-suspended in PBS to obtain a final volume of 100 μl. 20 μl of a secondary antibody (goat anti-mouse IgG Alexa-green) was added to each tube. After 30 minutes of incubation at 4° C., the stained cells were washed with PBS, re-suspended in 500 μl of 1% paraformaldehyde, and analyzed by FACS for EGFR expression.

Results:

pVL-CMV5-GFP and pVL-CR5-GFP baculovirus vectors were constructed and recombinant baculoviruses were generated and amplified using the Sf-9 insect cells. Transient gene expression using the baculoviruses was examined in Chinese Hamster Ovary (CHO) cell lines that stably express the cumate-modulated transactivator, rcTA. These cell lines are the CHOCymR-rcTA/10-35 cell line (Xu et al, 2004) in which expression is induced by cumate addition, and the CHO-rcTA/227 cell line in which rcTA expression is constitutively high resulting in cumate-independent reporter gene expression. Both cell lines are adapted for growth in suspension, in serum free medium.

In addition to the reporter gene GFP (for green fluorescent protein), the system was used successfully to express significant amounts of a kinase-competent human epidermal growth factor receptor (EGFR), and a Rep AAV protein that is toxic to most mammalian cells. The work described herein with baculovirus (BacMam) containing the CR5 promoter is aimed at enhancing baculovirus-mediated transient transgene expression in CHO cells. High level of protein expression obtained with the CR5 promoter offers the benefits of using the baculovirus at lower MOIs and thus offers a significant improvement for large scale protein production in CHO cells.

As opposed to protein production, where one cell type can be used to produce several proteins, for functional analysis it is important to use a relevant cell type for functional analysis of each protein. Making stable cell lines expressing the transactivator in each cell type of interest is feasible, but too cumbersome to be effective. It is therefore desirable to deliver both elements of the regulatory expression system, the reporter CR5-GFP and the activator rcTA, by baculovirus-mediated transduction. A baculovirus expressing rcTA was therefore generated under the control of a strong constitutive promoter, CMV5 (pVL-CMV5-rcTA). Strong expression and tight regulation in CHO cell lines using co-infection with the two baculoviruses was found.

The results herein show that the baculovirus expression system of the present invention, wherein transgene expression is regulated by a cumate-switch, offers a new and powerful tool for the expression of genes in mammalian cells. Given the strong activity of the CR5 promoter, lower MOIs can be used, thus decreasing the demand for very high titer stocks. Protein expression in mammalian cells may therefore be facilitated, be it for high-level production or functional analysis of the protein in question.

Expression of Green Fluorescent Protein (GFP):

The CHO cell line CHOCymR-rcTA/10-35 was transduced with BacMam-CMV5-GFP and BacMam-CR5-GFP baculoviruses. FIG. 1 shows the FACS data for GFP expression, with FIG. 1A showing the profile of the transduced cell population with respect to GFP expression and FIG. 1B quantifying the results. As expected, low GFP expression was observed in the cells transduced with BacMam-CR5-GFP in the absence of cumate (FIG. 1A: gray line; FIG. 1B: Fluorescence index (F.I.)=% of GFP positive cells×mean fluorescence intensity=212.5). Of note, however, is that this level (F.I.=212.5) is comparable to the fluorescence observed with CMV5-GFP (FIG. 1A: gray line; FIG. 1B: F.I.=244). Moreover, upon addition of cumate, GFP expression from BacMam-CR5-GFP increased 40-fold (FIG. 1A: black line; FIG. 1B: F.I.=9037). Treatment with butyrate further increased GFP expression 2.5-fold (FIG. 1A: filled area; FIG. 1B: F.I.=24,000). Expression from the CMV5 promoter was not significantly affected by cumate (FIG. 1A: solid line; FIG. 1B: F.I.=298), but was enhanced 3-fold by butyrate treatment (FIG. 1A: filled area; FIG. 1B: F.I.=780). However, even the butyrate-activated CMV5 promoter is 10-fold lower than the cumate-activated CR5 promoter and 25-fold lower than the cumate and butyrate-activated CR5 promoter. This demonstrates a significant and unexpected improvement in transgene expression using the BacMam expression system with the CR5 instead of with the CMV5 promoter.

Expression of a Toxic Protein, AVV Rep:

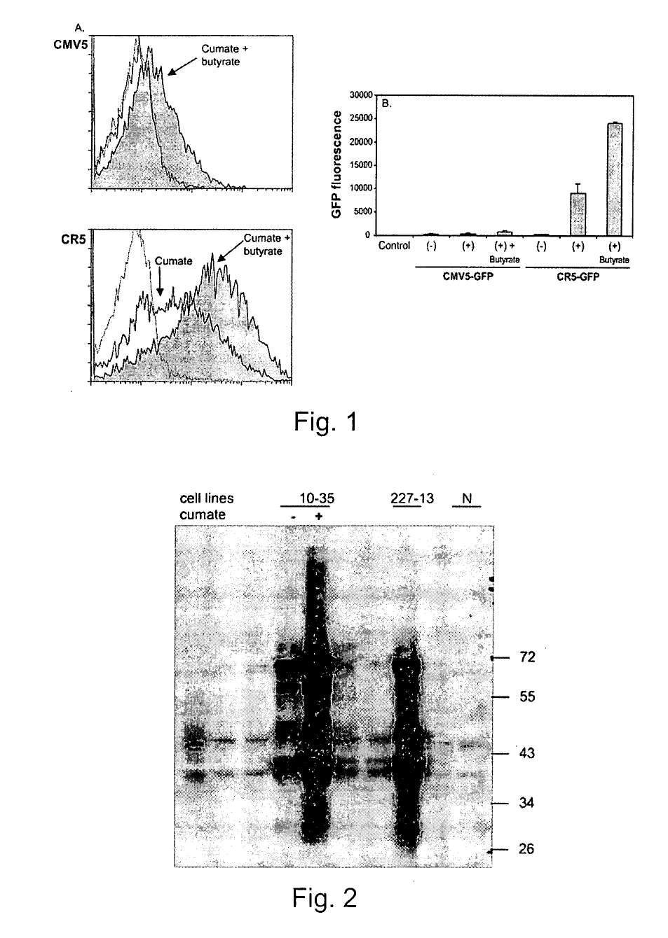

The single stranded DNA virus AAV uses four proteins for its replication function. These are Rep 78, 68, 52 and 40. In addition to their role in AAV replication, Rep 78 and Rep 52 inhibit transcription from adenoviral or helper virus genomes (Casto et al, 1967; Timp et al, 2006). Interestingly they do so by interfering with the function of cellular transcription factors that are required for adenoviral gene expression (Costello et al, 1997; Weger et al, 1999; Harmonat et al, 1998; Su et al, 2000; Prasad et al, 2003). The expression of these Rep proteins is therefore not very well tolerated by most mammalian cells, making any study requiring Rep expression a daunting task. Therefore, the Baculo-cumate system of the present invention was applied to develop an efficient Rep-expression system. FIG. 2 shows the result of a western analysis of CHOCymR-rcTA/10-35 (with and without cumate treatment) and CHO-rcTA/227 cells infected with BacMam-CR5-rep. Four major bands of the expected sizes are detected in extracts from CHOrcTA/10-35 and 227. In addition, cumate induction of Rep expression is evident in the CHOCymR-rcTA/10-35 cell line. Thus, the expression system of the present invention advantageously permits expression of the heretofore difficult to express AVV Rep proteins in mammalian cells.

Expression of Kinase-Competent EGFR:

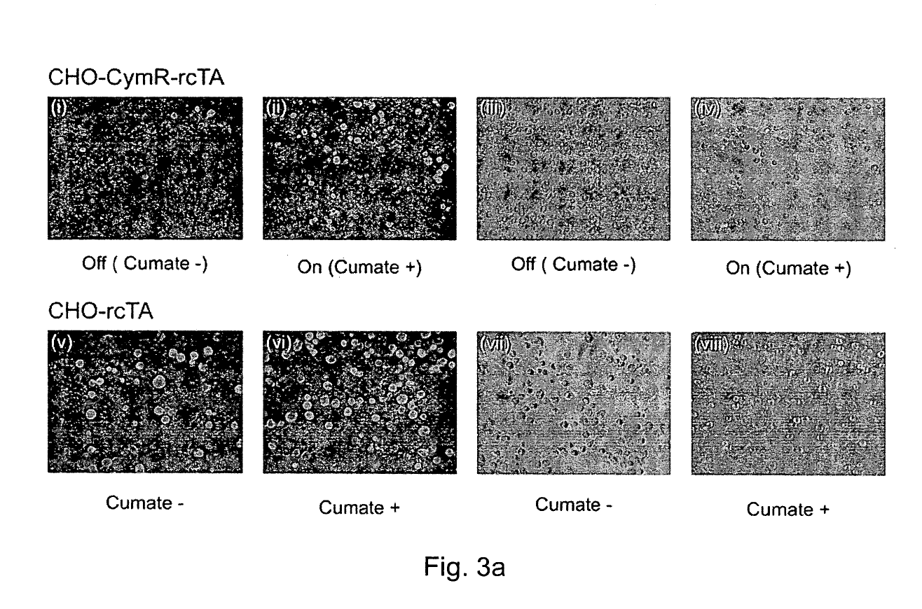

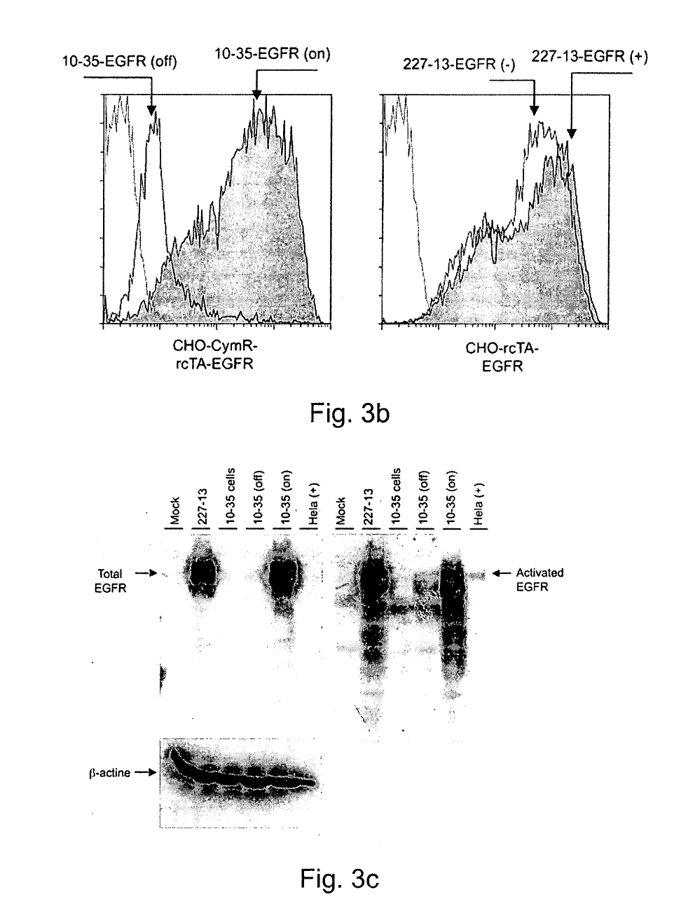

The epidermal growth factor receptor (EGFR) is a 170-kd transmembrane glycoprotein that mediates a range of biological responses of the epidermal growth factor. It is also being heavily investigated as a therapeutic target for several forms of cancer (Johnston et al, 2006). FIG. 3A shows the Alexa-green fluorescence (i, ii, v and vi) and phase (iii, iv, vii and viii) images of CHOCymR-rcTA/10-35 (i-iv) or CHO-rcTA/227-13 (v-viii) cells transduced with BacMam-CR5-EGFR, wherein EGFR expression was detected by virtue of binding to an Alexa-green-labelled antibody. The expression of EGFR is clearly detectable in both cell lines treated with cumate (ii and vi), but as expected, expression in CHOCymR-rcTA was significantly lower without the addition of cumate (compare i and ii).

A FACS profile of the transduced population is shown in FIG. 3B. In the case of CHOCymR-rcTA cells, the entire cell population is shifted from the negative profile (black line vs. gray line), however this shift is much more significant on cumate addition (filled peak). In the case of CHO-rcTA cells, transduction with BacMam-CR5-EGFR is sufficient to induce near-maximal expression of EGFR (black line versus filled profile). To test whether the EGFR molecule was kinase-competent, western blot analysis of the cell extracts was carried out using antibodies that detected either the total EGFR or the phosphorylated form. Extracts of EGF-stimulated HeLa cells served as a control for activated EGFR detection. FIG. 3C shows EGFR expression in CHOrcTA/10-35 cells transduced with BacMam-CR5-EGFR in the presence of cumate. Kinase activity of the EGFR is demonstrated in Panel B wherein the membrane has been probed with an antibody specific to the phosphorylated form of EGFR.

Co-transduction with BacMam-CR5-GFP and BacMam-CMV5-rcTA:

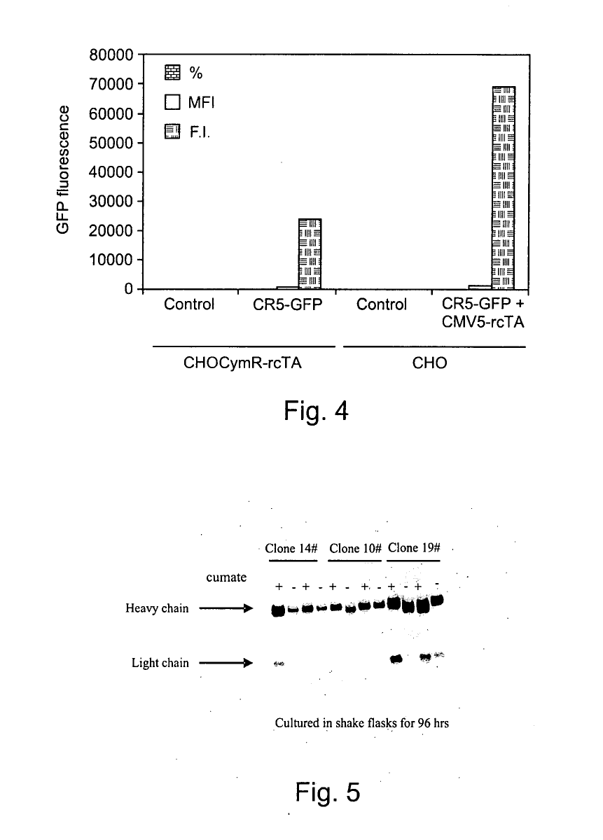

If the application for which a protein is being expressed involves the function of the protein, it is important that it be expressed in a cell type relevant for its function. A number of cell types that serve as models of physiological/pathological states are not amenable to transfection. Moreover, making a stable cell line expressing the activator is impractical. Therefore, an investigation was conducted to determine whether co-transduction with two baculoviruses, one expressing the activator and the other a reporter, could be a viable strategy to deliver the gene of interest in a relevant cell type. FIG. 4 compares GFP fluorescence in CHO cells co-transduced with BacMam-CR5-GFP (MOI=100) and BacMam-CMV5-rcTA (MOI=100) with those in CHOCymR-rcTA cells transduced with BacMam-CR5-GFP (MOI=100). Although both scenarios result in GFP expression in the majority of cells (88% GFP positive for CHOCymR-rcTA and 81.8% in the case of CHO), the intensity of expression is at least 3-fold (mean fluorescence intensity of 848 versus 268) higher in the BacMam-CR5-GFP and BacMam-CMV5-rcTA co-transduction, than in the CHOCymR-rcTA wherein rcTA is stably expressed in the host cell line. The most probable explanation for this difference is that the level of rcTA in CHOCymR-rcTA is limiting.

Expression of Antibody B43:

The results for the expression of the antibody B43 after transduction with the BacMam-CR5-b43 are shown in FIG. 5. The transduction was tested on three clones of the inducible cell line CHOCymR-rcTA/10-35, both heavy and light chains could be detected by probing with the anti-human IgG antibody. The clone #19 showed higher level of secretion. In addition the results with this clone show that in the absence of the inducer (cumate) the promoter activity was not detectable for the light chain but was clearly expressed up on addition of cumate. Promoter activity was detected for the heavy chain in both the presence and absence of cumate.

### REFERENCES

The disclosures in the references of the following list are herein incorporated by reference.
Boyce, F. M. and Bucher, N. L. R., 1996. Baculovirus-mediated gene transfer into mammalian cells. Proc. Natl. Acad. Sci. USA 93: 2348-2352.
Boyce, F. M., 1998. U.S. Pat. No. 5,731,182, issued Mar. 24, 1998.
Casto, B. C., Atchison, R. W., and Hammon, W. M., 1967. Studies on the relationship between adeno-associated virus type 1 (AAV1) and adenoviruses. Replication of AAV in certain cell cultures and its effect on helper adenoviruses. Virology 32: 52-59.
Condreay, P. J., Witherspoon, S. M., Clay, W. C and Kost, T. A., 1999. Transient and stable gene expression in mammalian cells transduced with a recombinant baculovirus vector. Proc. Natl. Acad. Sci. USA 96: 127-132.
Costello, E., Saudan, P., Winocour, E., Pizer, L., and Beard, P., 1997. High mobility group chromosomal protein 1 binds to the adeno-associated virus replication protein (Rep) and promotes Rep-mediated site-specific cleavage of DNA, ATPase activity and transcriptional repression. EMBO J. 16: 5943-5954.
Hermonat, P. L., Santin, A. D., Batchu, R. B., and Zhan, D., 1998. The adeno-associated virus Rep 78 major regulatory protein binds the cellular TATA-binding protein in vitro and in vivo. Virology 245: 120-127.
Hofmann, C., Sandig, V., Jennings, G., Rudolph, M., Schlag, P. and Strauss, M., 1995. Efficient gene transfer into human hepatocytes by baculovirus vectors. Proc. Natl. Acad. Sci. USA 92, 10099-10103.
Johnston, J. B., Navaratnam, S., Pitz, M. W., Maniate, J. M., Wiechec, E., Baust, H., Gingerich, J., Skliris, G. P., Murphy, L. C. and Los, M., 2006. Targeting the EGFR pathway for cancer therapy. Curr Med Chem. 13: 3483-92.
Kost, T. A. and Conderay, J. P., 2002. Recombinant baculoviruses as mammalian cell gene-delivery vectors. TRENDS in Biotechnology 20: 173-180.
Kost, T. A., Conderay, J. P. and Jarvis, D. L., 2005. Baculovirus as versatile vectors for protein expression in insect and mammalian cells. Nat. Biotechnol. 23: 567-75.
Massie, B., Mullick, A., Konishi, Y. and Lau, P., 2002. A cumate-inducible system for regulated expression in mammalian cells. US patent publication 2004/0205834 published Oct. 14, 2004.
Mullick, A., Xu, Y., Warren, R., Koutroumanis, M., Guilbault, C., Broussau, S., Malenfant, F., Bourget, L., Lamoureux, L., Lo, R., Caron, A. W., Pilotte, A. and Massie B., 2006. The cumate gene-switch: a system for regulated expression in mammalian cells. BMC Biotechnology 6:43.
Mullick, A; and Massie, B., 2000. Transcription, translation and the control of gene expression. In: The Encyclopedia of Cell Technology, Editor In Chief, Raymond E. Spei, Wiley Biotechnology Encyclopedias. pp 140-1164.
Xu, Y., Mullick, A., Massie, B., 2006. Efficient generation of a cumate-dependent gene switch system in eukaryotic cells through the mutations of p-Cym repressor. International patent publication WO 2006/037215 published Apr. 13, 2006.
Massie B., Mosser, D. D., Koutroumanis, M., Vitte-Mony, I., Lamoureux, L., Couture, F., Paquet, L., Guilbault, C., Dionne, J., Chahla, D. et al, 1998. New adenovirus vectors for protein production and gene transfer. Cytotechnology 28: 53-64.
Prasad, C. K., Meyers, C., Zhan, D. J., You, H., Chiriva-Internati, M., Mehta, J. L., Liu, Y., and Hermonat, P. L., 2003. The adeno-associated virus major regulatory protein Rep78-c-Jun-DNA motif complex modulates AP-1 activity. Virology 314: 423-431.
Ramos, L., Kopec, L. A., Sweitzer, S. M., Formwald, J. A., Zhao, H., Mcallister, P., McNulty, D. E., Trill, J. J. and Kane, J. F., 2002. Rapid expression of recombinant proteins in modified CHO cells using the baculovirus system. Cytotechnology 38: 37-41b.
Shen, C. F., Meghrous, J., Kamen, A., 2002. Quantitation of baculovirus particles by flow cytometry. Journal of Virological Methods 105: 321-330.
Smith, G. E., Summers, M. D., Fraser, M. J., 1983. Production of human beta interferon in insect cells infected with a baculovirus expression vector. Mol. Cell. Biol. 3: 2156-65.
Su, P. F., Chiang, S. Y., Wu, C. W., and Wu, F. Y., 2000. Adeno-associated virus major Rep 78 protein disrupts binding of TATA-binding protein to the p97 promoter of human papillomavirus type 16. J. Virol. 74: 2459-2465.
Timpe, J. M, Verrill K. C, Trempe, J. P., 2006. Effects of adeno-associated virus on adenovirus replication and gene expression during coinfection. J. Virol. 80: 7807-15.
Weger, S., Wendland, M., Kleinschmidt, J. A., and Heilbronn, R., 1999. The adeno-associated virus type 2 regulatory proteins Rep 78 and Rep 68 interact with the transcriptional coactivator PC4. J. Virol. 73: 260-269.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. An expression vector comprising a recombinant baculovirus operatively linked to a CR5 promoter.

2. The vector of claim 1, wherein the recombinant baculovirus is derived from pVL-1393.

3. A construct comprising the vector as defined in claim 1 operatively linked to a nucleic acid molecule of interest.

4. The construct of claim 3, wherein the nucleic acid molecule of interest lies 3′ to the CR5 promoter.

5. The construct of claim 3, wherein the nucleic acid molecule of interest codes for a protein.

6. A method of expressing a nucleic acid molecule of interest in a mammalian cell comprising transducing the mammalian cell by a construct as defined in claim 3 in the presence of an activator for the CR5 promoter.

7. The method of claim 6, wherein the activator is rcTA.

8. The method of claim 6, wherein the CR5 promoter is induced by an inducer.

9. The method of claim 8, wherein the inducer comprises cumate, butyrate, dimethyl-p-aminobenzoic acid, trimethyl cumate, ethylbenzoate, a salt thereof or a combination thereof.

10. The method of claim 8, wherein the inducer comprises cumate, butyrate or a combination thereof.

11. The method of claim 6, wherein the activator is expressed in the mammalian cell.

12. The method of claim 6, wherein the activator is introduced into the cell by a vector.

13. The method of claim 6, wherein the mammalian cell is a Chinese Hamster Ovary cell.

14. The method of claim 6, wherein the nucleic acid molecule of interest codes for a protein.

15. The method of claim 6, wherein the CR5 promoter is activated by rcTA, and wherein the mammalian cell is a Chinese Hamster Ovary cell, and wherein the nucleic acid molecule of interest codes for a protein.

16. The method of claim 15, wherein the recombinant baculovirus is derived from pVL-1393.

17. The method of claim 15, wherein the CR5 promoter is induced by cumate, butyrate or a combination thereof.

18. The method of claim 15, wherein the rcTA is expressed in the cell.

19. The method of claim 15, wherein the rcTA is introduced into the cell by a vector.

20. A host mammalian cell transduced by the construct of claim 3.

21. The cell of claim 20 which is a Chinese Hamster Ovary cell.

22. The cell of claim 20, wherein the nucleic acid molecule of interest codes for a protein, and wherein the CR5 promoter is activated by rcTA.

23. The cell of claim 22, wherein the recombinant baculovirus is derived from pVL-1393.
